(19) United States
(12) Patent Application Publication
(10) Pub. No.: US 2021/0012675 A1
(43) Pub. Date: Jan. 14, 2021

(54) TEACHING METHOD AND TEACHING DEVICE FOR IMPROVING ATTENTION, AND COMPUTER READABLE STORAGE MEDIUM

(71) Applicant: SHENZHEN MENTAL FLOW TECHNOLOGY CO., LTD., Shenzhen (CN)

(72) Inventors: Bicheng HAN, Shenzhen (CN); Hui ZHENG, Shenzhen (CN); Disi A, Shenzhen (CN); Dongsheng SUN, Shenzhen (CN); Zhaoyi YANG, Shenzhen (CN); Xiang YU, Shenzhen (CN); Yue SUN, Shenzhen (CN)

(73) Assignee: SHENZHEN MENTAL FLOW TECHNOLOGY CO., LTD., Shenzhen (CN)

(21) Appl. No.: 17/036,084

(22) Filed: Sep. 29, 2020

Related U.S. Application Data
(63) Continuation of application No. PCT/CN2019/084247, filed on Apr. 25, 2019.

(30) Foreign Application Priority Data
May 11, 2018 (CN) 201810447302.1

(51) Int. Cl. G09B 5/02; G06F 3/01; A61B 5/0476; A61B 5/16; A61B 5/04; G16H 50/30

(52) U.S. Cl. CPC G09B 5/02; G06F 3/015; G16H 50/30; A61B 5/168; A61B 5/04017; A61B 5/0476

(57) ABSTRACT

Disclosed are a teaching method and a teaching device for improving attention and a computer readable storage medium. The method includes the following operations: obtaining brainwave data of a user collected by an electroencephalograph (EEG) acquisition device, and calculating an attention value according to the brainwave data; switching to a training mode if the attention value is less than a first preset threshold; and obtaining an EEG feature of the user during training, and outputting and displaying an animation effect corresponding to the EEG feature to adjust the user's attention.

### CROSS-REFERENCE TO RELATED APPLICATIONS

This application is a continuation application of PCT Application No. PCT/CN2019/084247, filed on Apr. 25, 2019, which claims priority to Chinese application No. 201810447302.1, filed on May 11, 2018, entitled “TEACHING METHOD AND TEACHING DEVICE FOR IMPROVING ATTENTION, AND COMPUTER READABLE STORAGE MEDIUM”, the entire disclosure of which is incorporated herein by reference.

### TECHNICAL FIELD

The present disclosure relates to the technical field of education informatization, and in particular to a teaching method and a teaching device for improving attention and a computer readable storage medium.

### BACKGROUND

Electroencephalograph (EEG) signals accompany us throughout our lives, are the overall response of the spontaneous and rhythmic electrical activity of brain cells in the cerebral cortex and scalp, and can be detected by electrodes placed on the scalp. EEG can be divided into four rhythm waves: δ, θ, α, and β according to different frequencies. Through a large number of experiments, many foreign scholars and experts have discovered that the alpha band in human brainwaves is the main activity frequency in a quiet and awake state.

At present, the teaching system only includes playing rich media courseware or monitoring the level of students' attention, and does not train students to improve their attention in the classroom while monitoring their attention levels. Therefore, the current teaching system cannot improve the students' attention.

The above content is only used to assist the understanding of the technical solution of the present disclosure, and does not mean that the above content is recognized as prior art.

### SUMMARY

The main objective of the present disclosure is to provide a teaching method and a teaching device for improving attention, and a computer readable storage medium, aiming to solve the problem that the current teaching system cannot improve the students' attention.

In order to achieve the above objective, the present disclosure provides a teaching method for improving attention, including the following operations:

obtaining brainwave data of a user collected by an electroencephalograph (EEG) acquisition device, and calculating an attention value according to the brainwave data;

switching to a training mode if the attention value is less than a first preset threshold; and

obtaining an EEG feature of the user during training, and outputting and displaying an animation effect corresponding to the EEG feature to adjust the user's attention.

In an embodiment, the operation of obtaining an EEG feature of the user during training, and outputting and displaying an animation effect corresponding to the EEG feature to adjust the user's attention includes:

analyzing the EEG feature, and scoring the EEG feature according to a preset scoring rule to obtain a score;

comparing the score with a second preset threshold to obtain a comparison result; and

loading a file corresponding to the animation effect according to the comparison result, and playing a content of the file.

In an embodiment, the operation of analyzing the EEG feature, and scoring the EEG feature according to a preset scoring rule to obtain a score includes:

obtaining an Alpha wave, a Beta wave, a Delta wave, a Gamma wave and a Theta wave corresponding to the EEG feature;

calculating a mean value, a standard deviation, a ratio and a product of energy values corresponding to the Alpha wave, the Beta wave, the Delta wave, the Gamma wave and the Theta wave in a frequency domain to obtain the calculation result; and

scoring the EEG feature according to the calculation result and the preset scoring rule.

In an embodiment, after the operation of obtaining brainwave data of a user collected by an EEG acquisition device, the teaching method further includes:

removing center electricity, eye electricity and random noise of the brainwave data according to a first preset function to obtain data to be filtered; and

using a filter to filter the data to be filtered according to a second preset function, the filter being configured to remove low-frequency, high-frequency, and power frequency interference noise, and to separate rhythm waves in various frequency bands.

In an embodiment, after the operation of obtaining an EEG feature of the user during training, and outputting and displaying an animation effect corresponding to the EEG feature to adjust the user's attention, the teaching method further includes:

switching to a normal teaching mode if the training mode ends.

In an embodiment, the operation of switching to a training mode if the attention value is less than a first preset threshold includes:

sending a prompt for switching to the training mode to a management terminal if the attention value is less than the first preset threshold.

In an embodiment, the operation of switching to a training mode if the attention value is less than a first preset threshold further includes:

obtaining a break point corresponding to a current playing content if the attention value is less than the first preset threshold; and

automatically switching to the training mode at a time corresponding to the break point.

In an embodiment, the teaching method for improving attention further includes:

obtaining a training result of the user during the training mode;

sorting and storing the EEG feature, the attention value and the training result of the user during training; and

compressing and encrypting the EEG feature, the attention value and the training result, and generating an attention analysis report.

Besides, in order to achieve the above objective, the present disclosure further provides a teaching device for improving attention, including: a memory, a processor, a teaching program for improving attention stored on the memory and executable on the processor, the teaching program for improving attention, when executed by the processor, implements the operations of the teaching method for improving attention as described above.

In addition, in order to achieve the above objective, the present disclosure further provides a computer readable storage medium, a teaching program for improving attention is stored on the computer readable storage medium, the teaching program for improving attention, when executed by a processor, implements the operations of the teaching method for improving attention as described above.

In the present disclosure, brainwave data of a user collected by an EEG acquisition device is obtained, an attention value is calculated according to the brainwave data; it is switched to a training mode if the attention value is less than a first preset threshold; and an EEG feature of the user during training is obtained, and an animation effect corresponding to the EEG feature is output and displayed to adjust the user's attention. As a result, the user's attention is trained while monitoring the user's attention, thereby improving the user's attention.

### BRIEF DESCRIPTION OF THE DRAWINGS

FIG. 1 is a schematic structural diagram of a terminal to which a teaching device for improving attention belongs in a hardware operating environment according to an embodiment of the present disclosure.

FIG. 2 is a schematic flowchart of a teaching method for improving attention according to a first embodiment of the present disclosure.

FIG. 3 is a detailed schematic flowchart of the operation of obtaining an EEG feature of the user during training, and outputting and displaying an animation effect corresponding to the EEG feature to adjust the user's attention of the teaching method for improving attention according to a second embodiment of the present disclosure.

FIG. 4 is a detailed schematic flowchart of the operation of analyzing the EEG feature, and scoring the EEG feature according to a preset scoring rule to obtain a score of the teaching method for improving attention according to a third embodiment of the present disclosure.

FIG. 5 is a schematic flowchart of the teaching method for improving attention according to a fourth embodiment of the present disclosure.

FIG. 6 is a schematic flowchart of the teaching method for improving attention according to a fifth embodiment of the present disclosure.

FIG. 7 is a schematic flowchart of the operation of switching to a training mode if the attention value is less than a first preset threshold of the teaching method for improving attention according to a seventh embodiment of the present disclosure.

FIG. 8 is a schematic flowchart of the teaching method for improving attention according to an eighth embodiment of the present disclosure.

The realization of the objective, functional feature, and advantages of the present disclosure are further described with reference to the accompanying drawings.

### DETAILED DESCRIPTION OF THE EMBODIMENTS

It should be understood that the specific embodiments described herein are only used to explain the present disclosure, and are not intended to limit the present disclosure.

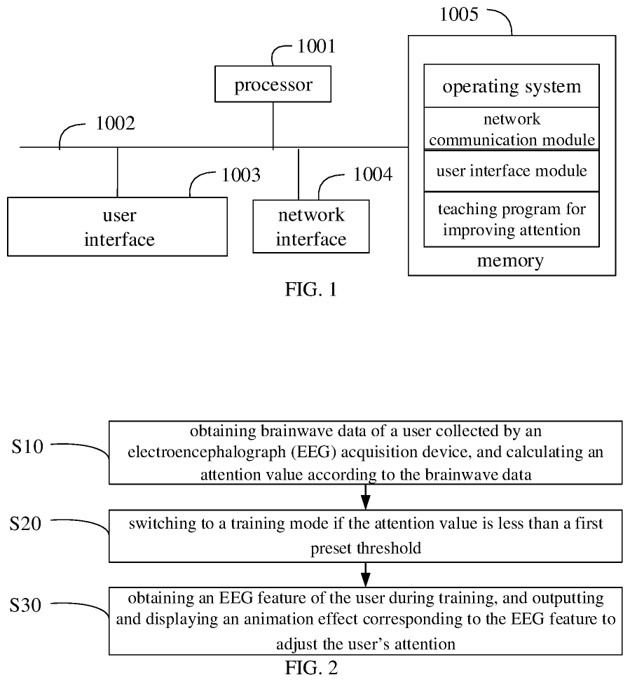

As shown in FIG. 1, FIG. 1 is a schematic structural diagram of a terminal in a hardware operating environment according to an embodiment of the present disclosure.

The terminal in the embodiment of the present disclosure may be a PC. As shown in FIG. 1, the terminal may include a processor 1001, such as a CPU, a network interface 1004, a user interface 1003, a memory 1005, and a communication bus 1002. The communication bus 1002 is configured to implement connection and communication between these components. The user interface 1003 may include a display, an input unit such as a keyboard. In an embodiment, the user interface 1003 may include a standard wired interface and a wireless interface. The network interface 1004 may include a standard wired interface and a wireless interface (such as a WI-FI interface). The memory 1005 may be a high-speed random access memory (RAM) memory or a non-volatile memory, such as a magnetic disk memory. The memory 1005 may be a storage device independent of the foregoing processor 1001.

In an embodiment, the terminal may also include a camera, a Radio Frequency (RF) circuit, a sensor, an audio circuit, a WiFi module, and so on. The sensors may be, for example, a light sensor, a motion sensor, and other sensors. Specifically, the light sensor may include an ambient light sensor and a proximity sensor. The ambient light sensor may adjust the brightness of the display according to the brightness of the ambient light. The proximity sensor may turn off the display and/or the backlight when the mobile terminal is moved to the ear. A gravity acceleration sensor, as a kind of motion sensor, may detect the magnitude of acceleration in various directions (usually three axes). The gravity acceleration sensor may detect the magnitude and direction of gravity when it is stationary, and may be configured to identify the gesture of the mobile terminal (such as horizontal and vertical screen switching, related games, magnetometer attitude calibration), vibration recognition related functions (such as pedometer, tap), etc. The mobile terminal may also be configured with other sensors such as a gyroscope, a barometer, a hygrometer, a thermometer, an infrared sensor, and the like, which will not be repeated here.

Those skilled in the art should understand that the terminal structure shown in FIG. 1 does not constitute a limitation on the terminal, and may include more or fewer components, a combination of some components, or differently arranged components than shown in the figure.

As shown in FIG. 1, the memory 1005 as a computer storage medium may include an operating server, a network communication module, a user interface module, and a program.

In the terminal shown in FIG. 1, the network interface 1004 is mainly configured to connect to a background server and perform data communication with the background server. The user interface 1003 is mainly configured to connect to a client (user terminal) and perform data communication with the client. The processor 1001 may be configured to call the program stored on the memory 1005.

In this embodiment, the device includes a memory 1005, a processor 1001, and a program stored on the memory 1005 and executable on the processor 1001, the processor 1001 performs the following operations when calling the program stored on the memory 1005:

obtaining brainwave data of a user collected by an electroencephalograph (EEG) acquisition device, and calculating an attention value according to the brainwave data;

switching to a training mode if the attention value is less than a first preset threshold; and

obtaining an EEG feature of the user during training, and outputting and displaying an animation effect corresponding to the EEG feature to adjust the user's attention.

Further, the processor 1001 can call a teaching program for improving attention stored on the memory 1005, and further perform the following operations:

analyzing the EEG feature, and scoring the EEG feature according to a preset scoring rule to obtain a score;

comparing the score with a second preset threshold to obtain a comparison result; and

loading a file corresponding to the animation effect according to the comparison result, and playing a content of the file.

Further, the processor 1001 can call a teaching program for improving attention stored on the memory 1005, and further perform the following operations:

obtaining an Alpha wave, a Beta wave, a Delta wave, a Gamma wave and a Theta wave corresponding to the EEG feature;

calculating a mean value, a standard deviation, a ratio and a product of energy values corresponding to the Alpha wave, the Beta wave, the Delta wave, the Gamma wave and the Theta wave in a frequency domain to obtain the calculation result; and

scoring the EEG feature according to the calculation result and the preset scoring rule.

Further, the processor 1001 can call a teaching program for improving attention stored on the memory 1005, and further perform the following operations:

removing center electricity, eye electricity and random noise of the brainwave data according to a first preset function to obtain data to be filtered; and

using a filter to filter the data to be filtered according to a second preset function, the filter being configured to remove low-frequency, high-frequency, and power frequency interference noise, and to separate rhythm waves in various frequency bands.

Further, the processor 1001 can call a teaching program for improving attention stored on the memory 1005, and further perform the following operations:

switching to a normal teaching mode if the training mode ends.

Further, the processor 1001 can call a teaching program for improving attention stored on the memory 1005, and further perform the following operations:

sending a prompt for switching to the training mode to a management terminal if the attention value is less than the first preset threshold.

Further, the processor 1001 can call a teaching program for improving attention stored on the memory 1005, and further perform the following operations:

obtaining a break point corresponding to a current playing content if the attention value is less than the first preset threshold; and

automatically switching to the training mode at a time corresponding to the break point.

Further, the processor 1001 can call a teaching program for improving attention stored on the memory 1005, and further perform the following operations:

obtaining a training result of the user during the training mode;

sorting and storing the EEG feature, the attention value and the training result of the user during training; and

compressing and encrypting the EEG feature, the attention value and the training result, and generating an attention analysis report.

The present disclosure further provides a teaching method for improving attention. As shown in FIG. 2, FIG. 2 is a schematic flowchart of the teaching method for improving attention according to a first embodiment of the present disclosure.

In this embodiment, the teaching method for improving attention includes:

Operation S10, obtaining brainwave data of a user collected by an EEG acquisition device, and calculating an attention value according to the brainwave data.

In this embodiment, the EEG acquisition device includes a headband that collects electroencephalograph (EEG). The headband can collect the user's brainwave data in real time. The brainwave data includes values corresponding to an Alpha wave, a Beta wave, a Delta wave, a Gamma waves and a Theta wave. The values of different frequency waves can reflect a current state of the human brain. For example, when people concentrate on studying, the brain frequency is in the Alpha wave (frequency range is 8 Hz to 13 Hz), and the brainwaves are relatively stable, which is the best brainwave state for people to study and think. When people are excited or nervous, the brain frequency is in the Beta wave (frequency range is greater than 14 Hz), and the brainwave frequency becomes faster and the amplitude increases. Appropriate Beta wave has a positive effect on the improvement of attention and the development of cognitive behavior, but the duration is short and people are easy to be fatigue. When people are tired and mentally relaxed, the brain frequency is in Theta wave.

Further, the collected brainwave data is sent to an attention training system. For example, when the data collection frequency is set to 160 HZ, 80 raw EEG data will be sent to the attention training system as a data packet every 0.5 seconds. The attention training system will calculate the user's current attention value according to the brainwave data. A machine learning training model can be used to calculate a predicted attention value, send the attention value to the display terminal, and monitor the attention value in real time.

Operation S20, switching to a training mode if the attention value is less than a first preset threshold.

In this embodiment, the first preset threshold is set by a technician. When in the normal teaching mode, the attention training system will detect the user's attention value in the normal teaching mode. When it is detected that the attention value is less than the first preset threshold, it will switch to the training mode. Further, if it is detected that the user's attention value is lower than a lower limit, it is determined that the user is not paying attention. If it is detected that the user's attention value is higher than an upper limit, it is determined that the user's attention is highly concentrated. If it is detected that the user's attention value is between the lower limit and the upper limit, it is determined that the user's attention is concentrated.

Furthermore, when the user is a whole class of students, the attention value of each student will be obtained, and an average attention value will be calculated, and the attention of the whole class will be determined according to the average attention value. Specifically, if the average attention value is less than a preset threshold, it is determined that the class is not paying attention. If the average attention value is greater than the preset threshold, it is determined that the class is focused. When the average attention value is less than the preset threshold, the normal teaching system is switched to the training system to enter the training mode.

Operation S30, obtaining an EEG feature of the user during training, and outputting and displaying an animation effect corresponding to the EEG feature to adjust the user's attention.

In this embodiment, the EEG feature includes delta, theta, alpha, beta, high-beta, and gamma frequency energy values, a mean value and a standard deviation of each frequency energy in the frequency domain, a ratio and a product of each frequency band energy. The EEG feature also includes the full frequency domain signal within the preset frequency range and the frequency domain feature of each frequency wave, for example, the full frequency domain signal below 80 Hz and the frequency domain feature of Theta wave, Alpha wave, Beta wave, Gamma wave and Theta wave band. The frequency domain feature includes a mean value, a peak value, and a standard deviation corresponding to the frequency wave. The EEG feature and frequency domain feature of each frequency wave are analyzed through the machine learning training model, and the weight of the EEG feature of each frequency wave is determined, and then the EEG feature are scored according to the weight and scoring rules.

Further, the user's attention can be trained through mini games, pictures and animation effects, for example, game programs such as flowers blooming, leaves growing, and diving. The human brain generates a small amount of electric current during its operation. The attention training system will detect the trainer's current brainwave activity and combine the actual situation of the brain to use designated computer games for the weak areas of the brain to help people exercise their brain nerves, thereby achieving the purpose of enhancing brain attention. During the training process, the user's EEG feature is scored, and the user is rewarded or punished through animation effects, thereby giving the user neurofeedback and improving the user's attention level.

This embodiment provides a teaching method for improving attention, including: obtaining brainwave data of a user collected by an electroencephalograph (EEG) acquisition device, and calculating an attention value according to the brainwave data; switching to a training mode if the attention value is less than a first preset threshold; and obtaining an EEG feature of the user during training, and outputting and displaying an animation effect corresponding to the EEG feature to adjust the user's attention. Thus, the user's attention is trained while monitoring the user's attention, thereby improving the user's attention.

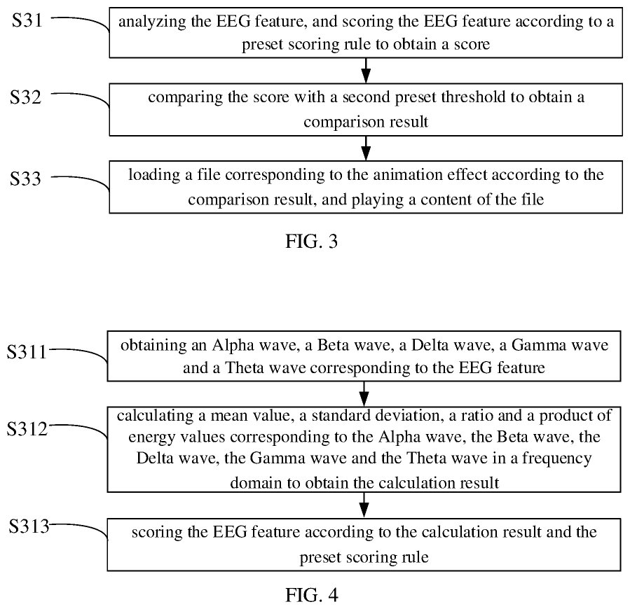

The present disclosure provides a second embodiment of the teaching method for improving attention according to the first embodiment. As shown in FIG. 3, in this embodiment, operation S30 includes:

Operation S31, analyzing the EEG feature, and scoring the EEG feature according to a preset scoring rule to obtain a score.

In this embodiment, analyzing the EEG feature includes calculating the mean, standard deviation, ratio and product of the energy values corresponding to the Alpha wave, Beta wave, Delta wave, Gamma wave and Theta wave in the frequency domain, and can also include calculating the percentage of the energy value corresponding to each frequency wave to the total energy, and can include calculating the energy value corresponding to the full frequency domain signal. The full frequency domain signal includes Alpha wave, Beta wave, Delta wave, Gamma wave, Theta wave and other frequency band signals. For example, analyzing the EEG feature includes calculating the total energy value corresponding to the energy value of Alpha wave, Beta wave, Delta wave, Gamma wave and Theta wave, then calculating the percentage of the energy value of Alpha wave, Beta wave, Delta wave, Gamma wave and Theta wave to the total energy value according to the total energy value.

Further, the preset scoring rule is set by a technician, and the attention training system can store a score table corresponding to the scores of the brainwave features, the scores of brainwave features in different ranges are different. For example, if the percentage of the energy value corresponding to the Alpha wave, Beta wave, Delta wave, Gamma wave and Theta wave to the total energy value is calculated, the calculated percentage is compared with the energy value percentage in the score ratio to determine the final score.

Operation S32, comparing the score with a second preset threshold to obtain a comparison result.

Operation S33, loading a file corresponding to the animation effect according to the comparison result, and playing a content of the file.

In this embodiment, the second preset threshold is set by a technician, and the score is compared with the second preset threshold. If the score is less than the second preset threshold, the file corresponding to the animation effect will be loaded and the content of the file will be played. For example, if the score is less than the second preset threshold, the animation effect file of the penalty theme will be loaded, and the content corresponding to the file will be played. If the score is greater than the second preset threshold, the animation effect file of the reward theme will be loaded, and the content corresponding to the file will be played. The average value corresponding to the EEG feature can be calculated according to the EEG feature of the whole class, and then it can be scored according to the average value.

This embodiment provides the teaching method for improving attention, including: analyzing the EEG feature, and scoring the EEG feature according to a preset scoring rule to obtain a score; comparing the score with a second preset threshold to obtain a comparison result; and loading a file corresponding to the animation effect according to the comparison result, and playing a content of the file. Therefore, the EEG feature can be scored and the corresponding animation effect can be displayed to improve students' attention.

The present disclosure provides a third embodiment of the teaching method for improving attention according to the second embodiment. As shown in FIG. 4, in this embodiment, operation S31 includes:

Operation 5311, obtaining an Alpha wave, a Beta wave, a Delta wave, a Gamma wave and a Theta wave corresponding to the EEG feature.

Operation 5312, calculating a mean value, a standard deviation, a ratio and a product of energy values corresponding to the Alpha wave, the Beta wave, the Delta wave, the Gamma wave and the Theta wave in a frequency domain to obtain the calculation result.

Operation 5313, scoring the EEG feature according to the calculation result and the preset scoring rule.

In this embodiment, the EEG feature includes delta, theta, alpha, beta, high-beta, and gamma frequency energy values, a mean value and a standard deviation of each frequency energy in the frequency domain, a ratio and a product of each frequency band energy. The mean value and the standard deviation of each frequency energy in the frequency domain, the ratio and the product of each frequency band energy can be selected. Any one or more of the ratios of in each frequency band energy is used as the EEG feature, and the percentage of the energy value corresponding to each frequency wave to the total energy value can also be calculated. For example, the total energy value corresponding to the energy value of Alpha wave, Beta wave, Delta wave, Gamma wave and Theta wave can be calculated, then the percentage of the energy value of Alpha wave, Beta wave, Delta wave, Gamma wave and Theta wave to the total energy value according to the total energy value can be calculated.

This embodiment provides the teaching method for improving attention, including: obtaining an Alpha wave, a Beta wave, a Delta wave, a Gamma wave and a Theta wave corresponding to the EEG feature; calculating a mean value, a standard deviation, a ratio and a product of energy values corresponding to the Alpha wave, the Beta wave, the Delta wave, the Gamma wave and the Theta wave in a frequency domain to obtain the calculation result; and scoring the EEG feature according to the calculation result and the preset scoring rule, thereby realizing scoring according to the EEG feature, and improving the user's attention.

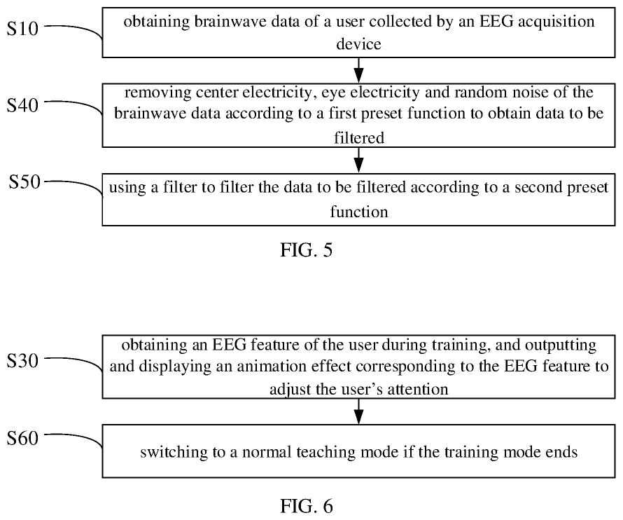

The present disclosure provides a fourth embodiment of the teaching method for improving attention according to the first embodiment. As shown in FIG. 5, in this embodiment, after operation S10, the method further includes:

Operation S40, removing center electricity, eye electricity and random noise of the brainwave data according to a first preset function to obtain data to be filtered.

In this embodiment, EEG signal is a kind of electrophysiological signal with strong randomness, and various emotions and mental states will affect its changes. Therefore, the EEG signal has a high time-varying sensitivity and is easily contaminated by irrelevant noise, thus forming various EEG artifacts. The most influential ones are ECG and ocular artifacts, electromyographic (EMG) signal interference caused by blinking, and potential changes caused by friction between the headband and the skin. The main feature of these noises are: special peaks on the frequency domain signal and the fractional frequency signal. The main function of the data preprocessing module is to detect and eliminate these noises. The data cleaning includes three operations, IMU action processing, blink detection, and crest compression. Specially, the IMU data is the physical movement data of the headband collected by the built-in module of the headband. The data includes an acceleration of the headband on the three coordinate axes of the three-dimensional space at this point-in-time. When the acceleration is greater than a threshold, it is determined that the data at that point-in-time is incredible. The incredible data segment is directly discarded, and linear interpolation is performed in the frequency domain. The first preset function is used to remove center electricity, eye electricity, and random noise of the brainwave data to obtain the data to be filtered. For example, V=V_{0} +(t−t_{0})*(V_{1}−V {0})/(t_{1}−t{0}). V is an interpolation at time t, V_{0} and V {1} are a start time and an end time of the discarded data segment, respectively, and V_{0} and V_{1} are voltage values at the corresponding time.

Operation S50, using a filter to filter the data to be filtered according to a second preset function, the filter being configured to remove low-frequency, high-frequency, and power frequency interference noise, and to separate rhythm waves in various frequency bands.

In this embodiment, the second preset function includes a butter function and a filtfilt function. The filter can be used to filter the data to be filtered. Band-pass filtering is performed first, and then band-stop filtering is performed. Low-frequency interference is mainly baseline drift, which is caused by poor contact between electrodes and human body, amplifier temperature drift or breathing during measurement. High-frequency interference is mainly radio frequency interference and EMG interference during acquisition. A Butterworth filter can be used for band-pass filtering, and the butter function and filtfilt function can be called to filter the filtered data. Further, a digital notch filter can be used to remove 50 Hz (or 60 Hz) power frequency interference, and a FIR digital filter can be used to separate various rhythm waves.

This embodiment provides the teaching method for improving attention, including: removing center electricity, eye electricity and random noise of the brainwave data according to a first preset function to obtain data to be filtered; and using a filter to filter the data to be filtered according to a second preset function, the filter being configured to remove low-frequency, high-frequency, and power frequency interference noise, and to separate rhythm waves in various frequency bands. Thus, the denoising and filtering of the brainwave data is realized, thereby ensuring the accuracy of detection.

The present disclosure provides a fifth embodiment of the teaching method for improving attention according to the fourth embodiment. As shown in FIG. 6, in this embodiment, after operation S30, the method further includes:

Operation S60, switching to a normal teaching mode if the training mode ends.

In this embodiment, if the training mode ends, it will automatically switch to the normal teaching mode, and the user's training result during the training mode will be stored and displayed on the terminal. The training result includes the energy values of frequency waves such as Alpha wave, Beta wave, Delta wave, Gamma wave and Theta wave, the EEG feature, the scores and the attention value corresponding to EEG feature during training. The user can see the change of attention in response to the change of brainwaves on the display terminal, and these data can be stored, so that the user's attention can be analyzed.

This embodiment provides the teaching method for improving attention, including: switching to a normal teaching mode if the training mode ends. Thus, it realizes the switching between training mode and normal teaching mode, which further improves the user's attention.

The present disclosure provides a sixth embodiment of the teaching method for improving attention according to the first embodiment. In this embodiment, operation S20 includes:

Operation S21, sending a prompt for switching to the training mode to a management terminal if the attention value is less than the first preset threshold.

In this embodiment, when the attention value is less than the first preset threshold, a prompt message for switching to the training mode is sent to the management terminal. The manager can use the management terminal to switch to the training mode, and select the scene corresponding to the training mode on the interface of the training mode, for example, you can select mini games or animation special effects, etc. When the manager clicks on the mini game in the training mode on the display interface of the management terminal for training, the scene corresponding to the mini game is displayed, and the user can test according to the mini game. The EEG acquisition device collects the user's brainwaves in real time. The EEG monitoring device monitors the change of the user's brainwaves in real time, and will warn in different forms on the software interface, and teachers can quickly see which students are not paying attention.

This embodiment provides the teaching method for improving attention, including: sending a prompt for switching to the training mode to a management terminal if the attention value is less than the first preset threshold. Thus, it is possible to prompt the manager to switch modes when the user's attention value is less than the first preset threshold, thereby improving the user experience.

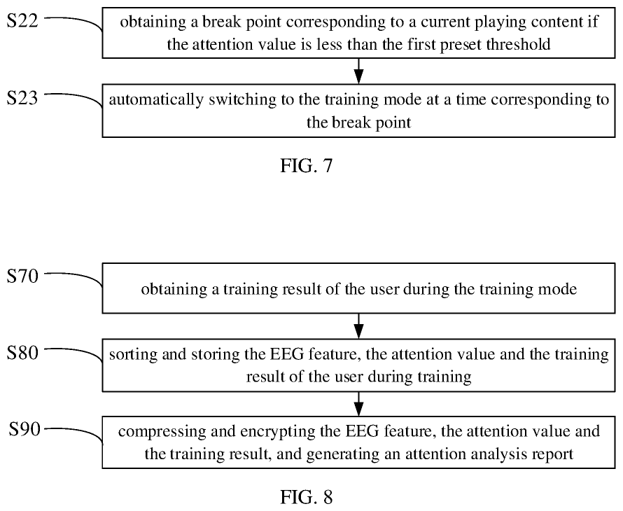

The present disclosure provides a seventh embodiment of the teaching method for improving attention according to the above embodiments. As shown in FIG. 7, in this embodiment, operation S20 further includes:

Operation S22, obtaining a break point corresponding to a current playing content if the attention value is less than the first preset threshold.

Operation S23, automatically switching to the training mode at a time corresponding to the break point.

In this embodiment, the break point can be set by a technician, the content of the normal mode teaching can be divided into many parts. The break point refers to the critical point between the various parts in the normal teaching mode. The location of the data corresponding to the critical point can be marked with a special mark, and each part of the content is connected by the break point, and each break point corresponds to a different time. If the attention is less than the first preset threshold, the time at which the current content is played is identified and the training mode is switched at that time. For example, the courseware content in the normal teaching mode is divided into 5 parts. When the teacher teaches the second part, if the attention value corresponding to the brainwaves is less than the first preset threshold, the break point corresponding to the part of the content is obtained, and the time corresponding to the break point is obtained, and the training mode is switched at that time.

This embodiment provides the teaching method for improving attention, including: obtaining a break point corresponding to a current playing content if the attention value is less than the first preset threshold; and automatically switching to the training mode at a time corresponding to the break point. Thus, the mode can be switched at the break point, thereby avoiding the confusion of mode switching, and further improving the teaching efficiency and quality.

The present disclosure provides an eighth embodiment of the teaching method for improving attention according to the seventh embodiment. As shown in FIG. 8, in this embodiment, the method further includes:

Operation S70, obtaining a training result of the user during the training mode.

Operation S80, sorting and storing the EEG feature, the attention value and the training result of the user during training.

Operation S90, compressing and encrypting the EEG feature, the attention value and the training result, and generating an attention analysis report.

In this embodiment, the EEG acquisition terminal sends the EEG feature, the training result, and the attention value of the user during training to the cloud server. The cloud server classifies and stores the data according to tags such as the user and the data collection environment, compresses and encrypts the data and stores it in the EEG database, and generates an attention analysis report. The cloud server sends the generated attention analysis report via email to the terminal where the student, teacher, parent and academic affairs office are located. The attention analysis report includes the attention value, a phase change curve of the attention value, the brainwave data, a curve graph analysis of characteristic data, etc. The cloud server can be replaced by redesigning Application Programming Interface (API).

Further, it also supports the entry and import of students' grades. Through the analysis of the historical data of the students' class situation and the students' learning achievements, the attention levels of different students, different classes, different courses, and different teachers are horizontally compared, the attention fluctuations of students or classes over a period of time are vertically compared, thereby providing methods and suggestions to improve students' learning efficiency. Teachers can personalize the seat layout on the detection terminal according to the actual situation of the classroom based on the number of students in the class and the learning situation of students, and connect the device terminal and display the connection status of the corresponding device terminal.

The present disclosure provides the teaching method for improving attention, including: obtaining a training result of the user during the training mode; sorting and storing the EEG feature, the attention value and the training result of the user during training; and compressing and encrypting the EEG feature, the attention value and the training result, and generating an attention analysis report. Therefore, the attention analysis report can be generated, which is convenient for users to understand the changes of students' attention in classroom teaching, which is conducive to the analysis of users' attention.

Besides, the present disclosure further provides a computer readable storage medium. The computer readable storage medium stores a teaching program for improving attention, the teaching program for improving attention, when executed by a processor, implements the following operations:

obtaining brainwave data of a user collected by an electroencephalograph (EEG) acquisition device, and calculating an attention value according to the brainwave data;

switching to a training mode if the attention value is less than a first preset threshold; and

obtaining an EEG feature of the user during training, and outputting and displaying an animation effect corresponding to the EEG feature to adjust the user's attention.

Further, the teaching program for improving attention, when executed by the processor, implements the following operations:

analyzing the EEG feature, and scoring the EEG feature according to a preset scoring rule to obtain a score;

comparing the score with a second preset threshold to obtain a comparison result; and

loading a file corresponding to the animation effect according to the comparison result, and playing a content of the file.

Further, the teaching program for improving attention, when executed by the processor, implements the following operations:

obtaining an Alpha wave, a Beta wave, a Delta wave, a Gamma wave and a Theta wave corresponding to the EEG feature;

calculating a mean value, a standard deviation, a ratio and a product of energy values corresponding to the Alpha wave, the Beta wave, the Delta wave, the Gamma wave and the Theta wave in a frequency domain to obtain the calculation result; and

scoring the EEG feature according to the calculation result and the preset scoring rule.

Further, the teaching program for improving attention, when executed by the processor, implements the following operations:

removing center electricity, eye electricity and random noise of the brainwave data according to a first preset function to obtain data to be filtered; and

using a filter to filter the data to be filtered according to a second preset function, the filter being configured to remove low-frequency, high-frequency, and power frequency interference noise, and to separate rhythm waves in various frequency bands.

Further, the teaching program for improving attention, when executed by the processor, implements the following operations:

switching to a normal teaching mode if the training mode ends.

Further, the teaching program for improving attention, when executed by the processor, implements the following operations:

sending a prompt for switching to the training mode to a management terminal if the attention value is less than the first preset threshold.

Further, the teaching program for improving attention, when executed by the processor, implements the following operations:

obtaining a break point corresponding to a current playing content if the attention value is less than the first preset threshold; and

automatically switching to the training mode at a time corresponding to the break point.

Further, the teaching program for improving attention, when executed by the processor, implements the following operations:

obtaining a training result of the user during the training mode;

sorting and storing the EEG feature, the attention value and the training result of the user during training; and

compressing and encrypting the EEG feature, the attention value and the training result, and generating an attention analysis report.

It should be noted that in this document, the terms “comprise”, “include” or any other variants thereof are intended to cover a non-exclusive inclusion. Thus, a process, method, article, or system that includes a series of elements not only includes those elements, but also includes other elements that are not explicitly listed, or also includes elements inherent to the process, method, article, or system. If there are no more restrictions, the element defined by the sentence “including a . . . ” does not exclude the existence of other identical elements in the process, method, article or system that includes the element.

The serial numbers of the foregoing embodiments of the present disclosure are only for description, and do not represent the advantages and disadvantages of the embodiments.

Through the description of the above embodiment, those skilled in the art can clearly understand that the above-mentioned embodiments can be implemented by software plus a necessary general hardware platform, of course, it can also be implemented by hardware, but in many cases the former is a better implementation. Based on this understanding, the technical solution of the present disclosure can be embodied in the form of software product in essence or the part that contributes to the existing technology. The computer software product is stored on a storage medium (such as ROM/RAM, magnetic disk, optical disk) as described above, including several instructions to cause a terminal device (which can be a mobile phone, a computer, a server, an air conditioner, or a network device, etc.) to execute the method described in each embodiment of the present disclosure.

The above are only some embodiments of the present disclosure, and do not limit the scope of the present disclosure thereto. Under the inventive concept of the present disclosure, equivalent structural transformations made according to the description and drawings of the present disclosure, or direct/indirect application in other related technical fields are included in the scope of the present disclosure.

What is claimed is:

1. A teaching method for improving attention, comprising the following operations:
obtaining brainwave data of a user collected by an electroencephalograph (EEG) acquisition device, and calculating an attention value according to the brainwave data;
switching to a training mode if the attention value is less than a first preset threshold; and
obtaining an EEG feature of the user during training, and outputting and displaying an animation effect corresponding to the EEG feature to adjust the user's attention.

2. The teaching method for improving attention of claim 1, wherein the operation of obtaining an EEG feature of the user during training, and outputting and displaying an animation effect corresponding to the EEG feature to adjust the user's attention comprises:
analyzing the EEG feature, and scoring the EEG feature according to a preset scoring rule to obtain a score;
comparing the score with a second preset threshold to obtain a comparison result; and
loading a file corresponding to the animation effect according to the comparison result, and playing a content of the file.

3. The teaching method for improving attention of claim 1, wherein the operation of analyzing the EEG feature, and scoring the EEG feature according to a preset scoring rule to obtain a score comprises:
obtaining an Alpha wave, a Beta wave, a Delta wave, a Gamma wave and a Theta wave corresponding to the EEG feature;
calculating a mean value, a standard deviation, a ratio and a product of energy values corresponding to the Alpha wave, the Beta wave, the Delta wave, the Gamma wave and the Theta wave in a frequency domain to obtain the calculation result; and
scoring the EEG feature according to the calculation result and the preset scoring rule.

4. The teaching method for improving attention of claim 1, wherein after the operation of obtaining brainwave data of a user collected by an EEG acquisition device, the teaching method further comprises:
removing center electricity, eye electricity and random noise of the brainwave data according to a first preset function to obtain data to be filtered; and
using a filter to filter the data to be filtered according to a second preset function, the filter being configured to remove low-frequency, high-frequency, and power frequency interference noise, and to separate rhythm waves in various frequency bands.

5. The teaching method for improving attention of claim 4, wherein after the operation of obtaining an EEG feature of the user during training, and outputting and displaying an animation effect corresponding to the EEG feature to adjust the user's attention, the teaching method further comprises:
switching to a normal teaching mode if the training mode ends.

6. The teaching method for improving attention of claim 1, wherein the operation of switching to a training mode if the attention value is less than a first preset threshold comprises:
sending a prompt for switching to the training mode to a management terminal if the attention value is less than the first preset threshold.

7. The teaching method for improving attention of claim 1, wherein the operation of switching to a training mode if the attention value is less than a first preset threshold further comprises:
obtaining a break point corresponding to a current playing content if the attention value is less than the first preset threshold; and
automatically switching to the training mode at a time corresponding to the break point.

8. The teaching method for improving attention of claim 7, further comprising:
obtaining a training result of the user during the training mode;
sorting and storing the EEG feature, the attention value and the training result of the user during training; and
compressing and encrypting the EEG feature, the attention value and the training result, and generating an attention analysis report.

9. A teaching device for improving attention, comprising a memory, a processor, a teaching program for improving attention stored on the memory and executable on the processor, the teaching program for improving attention, when executed by the processor, implements the following operations:
obtaining brainwave data of a user collected by an electroencephalograph (EEG) acquisition device, and calculating an attention value according to the brainwave data;
switching to a training mode if the attention value is less than a first preset threshold; and
obtaining an EEG feature of the user during training, and outputting and displaying an animation effect corresponding to the EEG feature to adjust the user's attention.

10. The teaching device for improving attention of claim 9, wherein the teaching program for improving attention, when executed by the processor, implements the following operations:
analyzing the EEG feature, and scoring the EEG feature according to a preset scoring rule to obtain a score;
comparing the score with a second preset threshold to obtain a comparison result; and
loading a file corresponding to the animation effect according to the comparison result, and playing a content of the file.

11. The teaching device for improving attention of claim 10, wherein the teaching program for improving attention, when executed by the processor, implements the following operations:
obtaining an Alpha wave, a Beta wave, a Delta wave, a Gamma wave and a Theta wave corresponding to the EEG feature;
calculating a mean value, a standard deviation, a ratio and a product of energy values corresponding to the Alpha wave, the Beta wave, the Delta wave, the Gamma wave and the Theta wave in a frequency domain to obtain the calculation result; and
scoring the EEG feature according to the calculation result and the preset scoring rule.

12. The teaching device for improving attention of claim 9, wherein the teaching program for improving attention, when executed by the processor, implements the following operations:
removing center electricity, eye electricity and random noise of the brainwave data according to a first preset function to obtain data to be filtered; and
using a filter to filter the data to be filtered according to a second preset function, the filter being configured to remove low-frequency, high-frequency, and power frequency interference noise, and to separate rhythm waves in various frequency bands.

13. The teaching device for improving attention of claim 12, wherein the teaching program for improving attention, when executed by the processor, implements the following operations:
switching to a normal teaching mode if the training mode ends.

14. The teaching device for improving attention of claim 9, wherein the teaching program for improving attention, when executed by the processor, implements the following operations:
sending a prompt for switching to the training mode to a management terminal if the attention value is less than the first preset threshold.

15. The teaching device for improving attention of claim 9, wherein the teaching program for improving attention, when executed by the processor, implements the following operations:
obtaining a break point corresponding to a current playing content if the attention value is less than the first preset threshold; and
automatically switching to the training mode at a time corresponding to the break point.

16. The teaching device for improving attention of claim 15, wherein the teaching program for improving attention, when executed by the processor, implements the following operations:
obtaining a training result of the user during the training mode;
sorting and storing the EEG feature, the attention value and the training result of the user during training; and
compressing and encrypting the EEG feature, the attention value and the training result, and generating an attention analysis report.

17. A computer readable storage medium, wherein a teaching program for improving attention is stored on the computer readable storage medium, the teaching program for improving attention, when executed by a processor, implements the following operations:
obtaining brainwave data of a user collected by an electroencephalograph (EEG) acquisition device, and calculating an attention value according to the brainwave data;
switching to a training mode if the attention value is less than a first preset threshold; and
obtaining an EEG feature of the user during training, and outputting and displaying an animation effect corresponding to the EEG feature to adjust the user's attention.

18. The computer readable storage medium of claim 17, wherein the teaching program for improving attention, when executed by the processor, implements the following operations:
analyzing the EEG feature, and scoring the EEG feature according to a preset scoring rule to obtain a score;
comparing the score with a second preset threshold to obtain a comparison result; and
loading a file corresponding to the animation effect according to the comparison result, and playing a content of the file.

19. The computer readable storage medium of claim 18, wherein the teaching program for improving attention, when executed by the processor, implements the following operations:
obtaining an Alpha wave, a Beta wave, a Delta wave, a Gamma wave and a Theta wave corresponding to the EEG feature;
calculating a mean value, a standard deviation, a ratio and a product of energy values corresponding to the Alpha wave, the Beta wave, the Delta wave, the Gamma wave and the Theta wave in a frequency domain to obtain the calculation result; and
scoring the EEG feature according to the calculation result and the preset scoring rule.

20. The computer readable storage medium of claim 17, wherein the teaching program for improving attention, when executed by the processor, implements the following operations:
removing center electricity, eye electricity and random noise of the brainwave data according to a first preset function to obtain data to be filtered; and
using a filter to filter the data to be filtered according to a second preset function, the filter being configured to remove low-frequency, high-frequency, and power frequency interference noise, and to separate rhythm waves in various frequency bands.
